(19) United States
(12) Patent Application Publication
(10) Pub. No.: US 2005/0191704 A1
(43) Pub. Date: Sep. 1, 2005

(54) Assay devices utilizing chemichromic dyes

(75) Inventors: RameshBabu Boga, Roswell, GA (US); John Gavin MacDonald, Decatur, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/790,617

(22) Filed: Mar. 1, 2004

(51) Int. Cl. G01N 33/53; C12Q 1/04; G01N 33/543

(52) U.S. Cl. 435/7.1; 435/34

(57) ABSTRACT

An assay device for detecting amines within a test sample (e.g., vaginal fluid) is provided. The assay device comprises a detection zone within which a chemichromic dye is contained. The chemichromic dye is capable of undergoing a color change upon exposure to one or more amines within the test sample.

### BACKGROUND OF THE INVENTION

The rapid diagnosis of infection is becoming increasingly important to improving the effectiveness of subsequent treatment. Vaginal infection (“vaginitis”), for example, exists in three primary forms, i.e., bacterial vaginosis, candidal vaginitis (“yeast”), and trichomonas vaginitis (“trich”). Various techniques have been developed in an attempt to rapidly diagnose the forms of vaginitis. For example, microbiological techniques have been utilized to identify “clue cells” (vaginal epithelial cells with adherent surface bacteria). However, conventional techniques for confirming the presence of “clue cells” are often complicated and slow. Likewise, techniques have been utilized that detect an elevated pH level in an infected sample. Unfortunately, conventional techniques for detecting an elevated pH level are often misleading due to other factors, such as the use of antimicrobials and cervical discharge, which also cause an elevated pH.

Still other techniques have been developed to diagnose vaginitis. For instance, some forms of vaginitis cause a “fishy” odor that stems from an elevated level of amines, such as putrescine (1, 4-diaminobutane), cadaverine (1, 5-diamino pentane), trimethylamine, etc., in an infected vaginal sample. In bacterial vaginosis, for instance, such amines are believed to be produced by members of anaerobic bacteria, prevotella, bacteroides, mobiluncus, and peptococcus. One conventional test for detecting the presence of amines in a vaginal test sample is known as the “Whiff test”, which involves adding a strong alkali to a sample to form an enhanced odor. Unfortunately, such tests are undesired in that they require performance by a professional and utilize caustic chemicals. Another conventional technique for detecting amines in a sample is described in U.S. Pat. No. 5,124,254 to Hewlins, et al. Hewlins, et al. uses a diamine oxidase that reacts with diamines, such as putrescine and cadaverine, to give hydrogen peroxide. The hydroxen peroxide is then detected by a chromogenic system. However, this technique is overly complex, costly, and time-consuming. Another problem with this technique is that it is inherently limited in that it is not able to detect other possible infection present in the test sample.

Apart from vaginitis, other types of infections also require rapid diagnosis. For example, many people (e.g., diabetics, burn victims, those suffering from suppressed immune systems, etc.) who have difficulty in healing and require extended periods for proper and complete wound healing are susceptible to infection. Bacteria and mold may also cause infection in hosts other than the human body, such as food. In many cases, these infections result in the formation of odorous amines and diamines, which may be produced by the metabolic processes of proteolytic bacteria together with short chain organic acids. Thus, as with vaginal infections, the ability to detect amines in other types contexts, such as in a wound exudate or food, would prove vastly beneficial.

As such, a need currently exists for a technique for detecting amines that is fast, inexpensive, and easy to use.

### SUMMARY OF THE INVENTION

In accordance with one embodiment of the present invention, an assay device for detecting the presence or absence of amines within a test sample is disclosed. The assay device comprises a fluidic medium (e.g., porous membrane, a flow channel, etc.) that defines a detection zone. Contained within the detection zone is a chemichromic dye that is capable of undergoing a detectable color change upon reaction with one or more amines. One particular example of a suitable chemichromic dye is an arylmethane, such as a diarylmethane or triarylmethane. In one embodiment, for example, the chemichromic dye is a triarylmethane having the following general structure:

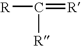

wherein R, R′, and R″ are independently selected from substituted and unsubstituted aryl groups. The aryl groups may be, for example, phenyl groups, naphthyl groups, or anthracenyl groups, and may be amino-substituted, hydroxyl-substituted, carboxyl-substituted, alkyl-substituted, sulfonic-substituted, carbonyl-substituted, or combinations thereof. Specific examples of such triarylmethanes include, but are not limited to, pararosanilin, alpha-naphtholbenzein, naphthocrome green, or analogs thereof. As stated, other arylmethanes are also suitable for use in the present invention. For example, in one embodiment, the chemichromic dye is a diarylmethane, such as 4,4′-bis (dimethylamino) benzhydrol or analogs thereof.

In some cases, the assay device is also capable of detecting the presence or absence of an analyte within the test sample. For example, the fluidic medium is in fluid communication with detection probes that are optionally conjugated with a specific binding member for the analyte. In addition, the fluidic medium may also define a second detection zone within which a capture reagent is immobilized. The capture reagent is configured to bind to the detection probes or complexes thereof to generate a detection signal, wherein the amount of an analyte in the test sample is proportional to the intensity of the detection signal.

In accordance with another embodiment of the present invention, an assay device for detecting the presence or absence of both amines and an analyte within a test sample is disclosed. The assay device comprises a porous membrane that is in fluid communication with detection probes conjugated with a specific binding for the analyte. The porous membrane defines a first detection zone within which a triarylmethane dye is immobilized. The triarylmethane dye is capable of undergoing a detectable color change upon reaction with one or more amines. The porous membrane also defines a second detection zone within a capture reagent is immobilized. The capture reagent is configured to bind to the detection probes or complexes thereof to generate a detection signal. The amount of an analyte in the test sample is proportional to the intensity of the detection signal.

In accordance with still another embodiment of the present invention, a method for detecting the presence or absence of amines within a test sample is disclosed. The method comprises contacting an assay device with a test sample containing one or more amines. The assay device comprises a fluidic medium that defines a detection zone, wherein a chemichromic dye is contained within the detection zone that undergoes a color change upon reacting with the amines. The method further comprises measuring the color intensity of the chemichromic dye at the detection zone after reacting with the amines, wherein the color intensity corresponds to a certain concentration of the amines within the test sample. In some cases, this color intensity may also be compared to the color intensity of a chemichromic dye that is not reacted with amines. This dye, which is not reacted with amines, may be contained within a control zone defined by the fluidic medium.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a perspective view of one embodiment of a flow-through assay device of the present invention;

FIG. 2 is a perspective view of another embodiment of a flow-through assay device of the present invention;

FIG. 3 is a schematic illustration of the mechanism used for one embodiment of the present invention;

FIG. 4 is a graphical illustration of the detection curve generated for Example 1 in which absorbance is plotted versus known putrescine concentrations using alpha-naphtholbenzein as the chemichromic dye;

FIG. 5 is a graphical illustration of the detection curve for Example 2 in which absorbance is plotted versus known cadaverine concentrations using alpha-naphtholbenzein as the chemichromic dye;

FIG. 6 is a graphical illustration of the detection curve for Example 3 in which absorbance is plotted versus known trimethylamine concentrations using alpha-naphtholbenzein as the chemichromic dye; and

FIG. 7 is a graphical illustration of the detection curve generated for Example 5 in which reflectance measurements are plotted versus known putrescine concentrations using alpha-naphtholbenzein as the chemichromic dye.

Repeat use of reference characters in the present specification and drawings is intended to represent same or analogous features or elements of the invention.

### DETAILED DESCRIPTION OF REPRESENTATIVE EMBODIMENTS

### Definitions

As used herein, the term “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include ferritin; creatinine kinase MB (CK-MB); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; candida albicans; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Other potential analytes may be described in U.S. Pat. No. 6,436,651 to Everhart, et al. and U.S. Pat. No. 4,366,241 to Tom et al.

As used herein, the term “test sample” generally refers to a biological material suspected of containing the analyte. The test sample may be obtained or derived from any biological source, such as a physiological fluid, including, blood, interstitial fluid, saliva, ocular lens fluid, cerebral spinal fluid, sweat, urine, milk, ascites fluid, mucous, synovial fluid, peritoneal fluid, vaginal fluid, amniotic fluid, and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, inactivation of interfering components, the addition of reagents, etc. Moreover, it may also be beneficial to modify a solid test sample to form a liquid medium or to release the analyte.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

In general, the present invention is directed to an assay device for detecting the presence of amines (monoamines, diamines, and/or tertiary amines) in a test sample. Specifically, the assay device includes a detection zone within which is contained a chemichromic dye, i.e., a dye that exhibits a detectable color change upon chemical reaction with one or more functional groups, such as amino groups. The assay device is also multi-functional in that it is capable of simultaneously detecting the presence of an analyte within the test sample.

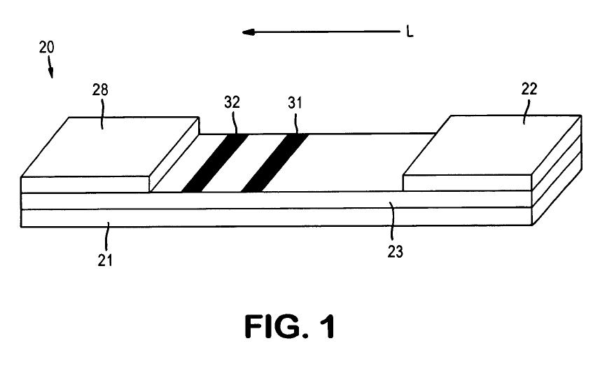

Referring to FIG. 1, for instance, one embodiment of a membrane-based flow-through assay device 20 that may be formed according to the present invention will now be described in more detail. As shown, the device 20 contains a porous membrane 23 that acts as a fluidic medium and is optionally supported by a rigid material 21. In general, the porous membrane 23 may be made from any of a variety of materials through which the test sample is capable of passing. For example, the materials used to form the porous membrane 23 may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the porous membrane 23 is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The device 20 may also contain an absorbent pad 28. The absorbent pad 28 generally receives fluid that has migrated through the entire porous membrane 23. As is well known in the art, the absorbent pad 28 may assist in promoting capillary action and fluid flow through the membrane 23.

To initiate the detection of amines within the test sample, a user may directly apply the test sample to a portion of the porous membrane 23 through which it may then travel. Alternatively, the test sample may first be applied to a sampling pad (not shown) that is in fluid communication with the porous membrane 23. Some suitable materials that may be used to form the sampling pad include, but are not limited to, nitrocellulose, cellulose, porous polyethylene pads, and glass fiber filter paper. If desired, the sampling pad may also contain one or more assay pretreatment reagents, either diffusively or non-diffusively attached thereto. In the illustrated embodiment, the test sample travels from the sampling pad (not shown) to a conjugate pad 22 that is placed in communication with one end of the sampling pad. The conjugate pad 22 is formed from a material through which the test sample is capable of passing. For example, in one embodiment, the conjugate pad 22 is formed from glass fibers. Although only one conjugate pad 22 is shown, it should be understood that other conjugate pads may also be used in the present invention.

Because the conjugate pad 22 is in fluid communication with the porous membrane 23, the test sample may migrate from the conjugate pad 22 to a detection zone 31 defined by the porous membrane 23 that is capable of signaling the presence of an amine. In particular, the detection zone 31 includes a “chemichromic dye”, i.e., a dye that exhibits a detectable color change upon chemical reaction with one or more functional groups. Without intending to be limited by theory, it is believed that the addition of an amino functional group (NH₂) to the chemichromic dye molecule induces either a shift of the absorption maxima towards the red end of the spectrum (“bathochromic shift”) or towards the blue end of the spectrum (“hypsochromic shift”). The type of absorption shift depends on the nature of the dye molecule and on whether the amino group functions as an electron acceptor (oxidizing agent), in which a hypsochromic shift results, or whether the amino group functions as an electron donor (reducing agent), in which a bathochromic shift results. Regardless, the absorption shift provides a color difference that is detectable, either visually or through instrumentation, to indicate the presence of amines in the test sample. For example, prior to contact with an infected test sample, the chemichromic dye may be colorless or it may possess a certain color. However, after contacting the test sample and reacting with amines present therein, the dye exhibits a change in color that is different than its initial color. That is, the dye may change from a first color to a second color, from no color to a color, or from a color to no color.

Generally speaking, any chemichromic dye capable of exhibiting a detectable change in color upon reaction with an amine may be utilized in the present invention. Such dyes are generally well known to those skill in the art, and may be described, for instance, in U.S. Pat. No. 4,477,635 to Mitra; U.S. Pat. No. 5,837,429 to Nohr, et al.; U.S. Pat. No. 6,174,646 to Hirai, et al., which are incorporated herein in their entirety by reference thereto for all purposes. For example, one class of chemichromic dyes that is particularly useful in the present invention is arylmethane dyes, such as diarylmethanes, triarylmethanes, and so forth.

Triarylmethane dyes, for example, may have the following general structure: 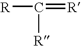
wherein R, R′, and R″ are independently selected from substituted and unsubstituted aryl groups, such as phenyl, naphthyl, anthracenyl, etc. The aryl groups may, for example, be substituted with functional groups, such as amino, hydroxyl, carbonyl, carboxyl, sulfonic, alkyl, and/or other known functional groups. When contacted with the dye, the amino group of the amine (e.g., ammonia, diamines, and/or tertiary amines) reacts with the central carbon atom of the dye. The addition of the amino group causes the dye to undergo a change in color. An example of the resulting structure is set forth below:

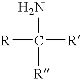

One particular example of a suitable triarylmethane dye is pararosanilin (also known as “basic fuchsin” or “magenta 0”) and analogs thereof, such as rosanilin (“magenta I”), magenta II, new fuchsin (“magenta III”), methyl violet 2B, methyl violet 6B, methyl violet 10B (“crystal violet”), methyl green, ethyl green, acid fuchsin, and so forth. Pararosanilin shifts from a red color to colorless (i.e., white) upon reaction with an amine. Pararosanilin contains three phenylamine groups (i.e., amino-substituted aryl groups). Specifically, the structure of the structure of pararosanilin is set forth below:

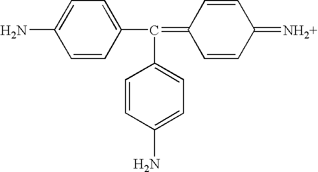

In some cases, triarylmethane dyes may be formed by converting a leuco base to a colorless carbinol and then treating the carbinol with an acid to oxidize the carbinol and form the dye. Thus, for example, pararosanilin may be derived by reacting the carbinol form of pararosanilin (“pararosaniline base”) with an acid, such as, but not limited to, sulfonic acids, phosphoric acids, hydrochloric acid, and so forth. The carbinol form of pararosanilin is set forth below.

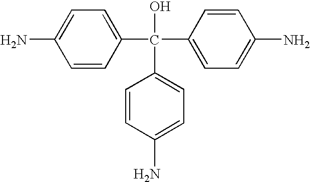

Another example of a suitable triarylmethane dye is alpha-naphtholbenzein and analogs thereof. Alpha-naphtholbenzein turns from an orange/red color to a gray/black color upon reaction with an amine. Alpha-naphtholbenzein contains a hydroxyl-substituted naphthyl group, a carbonyl-substituted naphthyl group, and a phenyl group. Specifically, the structure of alpha-naphtholbenzein is set forth below:

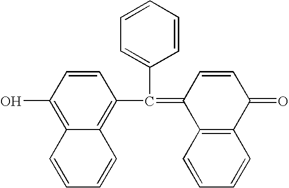

Still another example of a suitable triarylmethane dye is naphthocrome green and analogs thereof. Naphthocrome green turns from a pale yellow color to a blue/green color upon reaction with an amine. Similar to alpha-naphtholbenzein, naphthocrome green contains a hydroxyl-substituted naphthyl group, a carbonyl-substituted naphthyl group, and a phenyl group. However, each naphthyl group is also substituted with a sodium carboxyl. Specifically, the structure of naphthocrome green is set forth below:

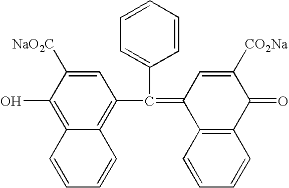

As indicated above, diarylmethanes may also be used in the present invention. One example of such a diarylmethane is 4,4′-bis (dimethylamino) benzhydrol (also known as “Michler's hydrol”), which has the following structure:

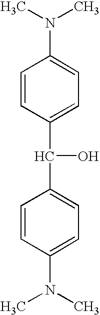

Still other examples include analogs of Michler's hydrol, such as Michler's hydrol leucobenzotriazole, Michler's hydrol leucomorpholine, Michler's hydrol leucobenzenesulfonamide, and so forth, as well as other diarylmethanes, such as malachite green leuco, malachite green carbinol, sodium 2,6-dichloroindopheno-late, rhodamine lactam, crystal violet lactone, and crystal violet leuco.

Regardless of the dye selected, any of a variety of techniques may be employed to apply the dye to the porous membrane 23. The dyes may be applied directly to the membrane 23 or first formed into a solution prior to application. Various solvents may be utilized to form the solution, such as, but not limited to, acetonitrile, dimethylsulfoxide (DMSO), ethyl alcohol, dimethylformamide (DMF), and other polar organic solvents. The amount of the dye in the solution may range from about 0.001 to about 1 milligram per milliliter of solvent, and in some embodiments, from about 0.01 to about 0.1 milligrams per milliliter of solvent. The dye solution may be coated onto the porous membrane 23 using well-known techniques and then dried. The dye concentration may be selectively controlled to provide the desired level of detection sensitivity. For example, higher concentrations may provide a higher level of detection sensitivity when low amine levels are suspected.

In some cases, the chemichromic dye is applied in a manner so that it does not substantially diffuse through the matrix of the porous membrane 23. This enables a user to readily detect the change in color that occurs upon reaction of the dye with an amine. For instance, the chemichromic dye may form an ionic and/or covalent bond with functional groups present on the surface of the porous membrane 23 so that it remains immobilized thereon. For example, in one embodiment, a positively-charged chemichromic dye may form an ionic bond with negatively-charged carboxyl groups present on the surface of some porous membranes (e.g., nitrocellulose). In other embodiments, the use of particles may facilitate the immobilization of the chemichromic dye at the detection zone 31. Namely, the dye may be coated onto particles (sometimes referred to as “beads” or “microbeads”) that are then immobilized on the porous membrane 23 of the assay device 20. In this manner, the dye is able to readily contact a test sample flowing through the membrane 23. For instance, naturally occurring particles, such as nuclei, mycoplasma, plasmids, plastids, mammalian cells (e.g., erythrocyte ghosts), unicellular microorganisms (e.g., bacteria), polysaccharides (e.g., agarose), and so forth, may be used. Further, synthetic particles may also be utilized. For example, in one embodiment, latex particles may be labeled with the chemichromic dye. Although any latex particle may be used in the present invention, the latex particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable particles may be described in U.S. Pat. No. 5,670,381 to Jou, et al. and U.S. Pat. No. 5,252,459 to Tarcha, et al., which are incorporated herein in their entirety by reference thereto for all purposes.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 1,000 microns, in some embodiments, from about 0.1 nanometers to about 100 microns, and in some embodiments, from about 1 nanometer to about 10 microns. For instance, “micron-scale” particles are often desired. When utilized, such “micron-scale” particles may have an average size of from about 1 micron to about 1,000 microns, in some embodiments from about 1 micron to about 100 microns, and in some embodiments, from about 1 micron to about 10 microns. Likewise, “nano-scale” particles may also be utilized. Such “nano-scale” particles may have an average size of from about 0.1 to about 10 nanometers, in some embodiments from about 0.1 to about 5 nanometers, and in some embodiments, from about 1 to about 5 nanometers.

Although non-diffusive immobilizing techniques may be desired in some cases, it should also be understood that any other technique for applying the chemichromic dye to the porous membrane 23 may be used in the present invention. In fact, the aforementioned methods are only intended to be exemplary of the techniques that may be used in the present invention. For example, in some embodiments, certain components may be added to a chemichromic dye solution that substantially inhibit the diffusion of the dye into the matrix of the porous membrane 23. In other cases, immobilization may not be required, and the dye may instead diffuse into the matrix of the porous membrane 23 for reaction with the test sample.

The detection zone 31 may generally provide any number of distinct detection regions so that a user may better determine the concentration of a particular analyte within a test sample. Each region may contain the chemichromic dye, or may contain different dyes for reacting with different types of amines. For example, the detection zone 31 may include two or more distinct detection regions (e.g., lines, dots, etc.). The detection regions may be disposed in the form of lines in a direction that is substantially perpendicular to the flow of the test sample through the assay device 20. Likewise, in some embodiments, the detection regions may be disposed in the form of lines in a direction that is substantially parallel to the flow of the test sample through the assay device.

If desired, the assay device 20 may employ a control zone 32 that is applied with the same chemichromic dye applied to the detection zone 31 and positioned downstream from the detection zone 31. In addition, the detection zone 31 may be provided with an amount of dye that is equal to or in excess of the amount needed to fully react with all of the amines present within the test sample. Thus, amines from the test sample will react only at the detection zone 31 and not at the control zone 32. In this manner, the color of the control zone 32 will generally remain unchanged so that it may be compared to the color of the detection zone 31 for determining the extent to which it changed after reaction with the amines. Similar to the detection zone 31, the control zone 32 may also provide any number of distinct regions:

After allowing the test sample to react for a sufficient time, the color of the detection zone 31 and/or control zone 32 may be determined either visually or using instrumentation. If desired, the intensity of the color at the zones 31 and/or 32 may be measured to quantitatively or semi-quantitatively determine the level of amines present in the test sample. In one embodiment, color intensity is measured as a function of absorbance, with an increased absorbance generally representing an increased amine concentration. For example, absorbance readings may be measured at a wavelength of 650 nanometers using a microplate reader from Dynex Technologies of Chantilly, Va. (Model # MRX).

In another embodiment, color intensity may be measured using a conventional test known as “CIELAB”, which is discussed in Pocket Guide to Digital Printing by F. Cost, Delmar Publishers, Albany, N.Y. ISBN 0-8273-7592-1 at pages 144 and 145. This method defines three variables, L*, a*, and b*, which correspond to three characteristics of a perceived color based on the opponent theory of color perception. The three variables have the following meaning:
L*=Lightness, ranging from 0 to 100, where 0 =dark and 100=light;
a*=Red/green axis, ranging approximately from −100 to 100; positive values are reddish and negative values are greenish; and
b*=Yellow/blue axis, ranging approximately from −100 to 100; positive values are yellowish and negative values are bluish.

Because CIELAB color space is somewhat uniform, a single number may be calculated that represents the difference between two colors as perceived by a human. This difference is termed ΔE and calculated by taking the square root of the sum of the squares of the three differences (ΔL*, Δa*, and Δb*) between the two colors. In CIELAB color space, each ΔE unit is approximately equal to a “just noticeable” difference between two colors. CIELAB is therefore a good measure for an objective device-independent color specification system that may be used as a reference color space for the purpose of color management and expression of changes in color. Using this test, color intensities (L*, a*, and b*) may thus be measured using, for instance, a handheld spectrophotometer from Minolta Co. Ltd. of Osaka, Japan (Model # CM2600d). This instrument utilizes the D/8 geometry conforming to CIE No. 15, ISO 7724/1, ASTME1164 and JIS Z8722-1982 (diffused illumination/8-degree viewing system. The D65 light reflected by the specimen surface at an angle of 8 degrees to the normal of the surface is received by the specimen-measuring optical system. Still other suitable devices for measuring the intensity of a visual color may also be used in the present invention. For example, one suitable reflectance reader is described in U.S. Patent App. Pub. No. 2003/0119202 to Kaylor, et al., which is incorporated herein in its entirety by reference thereto for all purposes.

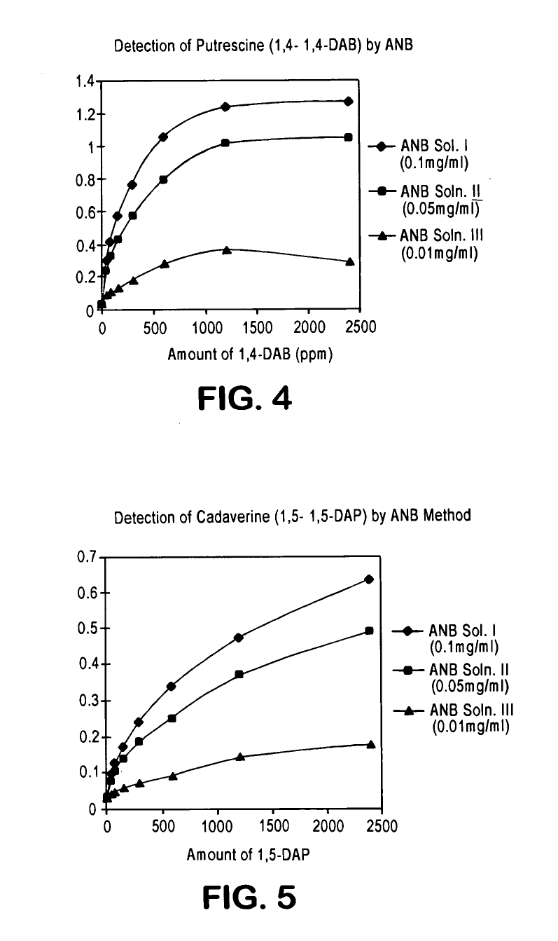

Regardless of the manner in which color intensity is measured, the result may be compared with a predetermined detection curve in which the color of the reacted chemichromic dye is plotted versus various known concentrations of an amine. In this manner, the color of the dye may be measured after reacting with a test sample and readily correlated to an amine concentration for providing quantitative or semi-quantitative results to a user. For instance, FIGS. 4-7 illustrate example detection curves for measuring the amount of putrescine, cadaverine, and trimethylamine in a test sample with an alpha-naphtholbenzein chemichromic dye. As is well known in the art, the amount of the dye present within the detection zone 31 may be tailored to be equal to or in excess of the maximum amount of suspected amines within the test sample.

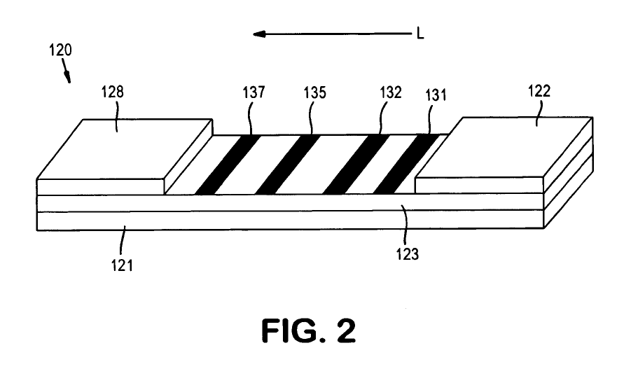

Besides detecting the presence of amines in a test sample, the assay device of the present invention is also able to detect the presence of an analyte. In this manner, for example, vaginal fluid may be simultaneously tested for the presence of amines and also for the presence of other diseases or disorders. Referring to FIG. 2, for example, one embodiment of an assay device 120 is shown that is configured to simultaneously detect the presence of an analyte and amines within a test sample. Similar to the assay device 20 of FIG. 1, the assay device 120 contains a porous membrane 123 optionally supported by a rigid material 121. The assay device 120 also contains a sampling pad (not shown), a conjugate pad 122, and an absorbent pad 128 in fluid communication with the porous membrane 123.

To facilitate accurate detection of an analyte within the test sample, a predetermined amount of detection probes may be applied at various locations of the device 120, such as to a conjugate pad 122. Any substance generally capable of producing a signal that is detectable visually or by an instrumental device may be used as detection probes. Various suitable substances may include calorimetric or fluorescent chromogens; catalysts; luminescent compounds (e.g., fluorescent, phosphorescent, etc.); radioactive compounds; visual labels, including colloidal metallic (e.g., gold) and non-metallic particles, dyed particles, hollow particles, enzymes or substrates, or organic polymer latex particles; liposomes or other vesicles containing signal producing substances; and so forth. For instance, some enzymes suitable for use as detection probes are disclosed in U.S. Pat. No. 4,275,149 to Litman. et al., which is incorporated herein in its entirety by reference thereto for all purposes. One example of an enzyme/substrate system is the enzyme alkaline phosphatase and the substrate nitro blue tetrazolium-5-bromo-4-chloro-3-indolyl phosphate, or derivative or analog thereof, or the substrate 4-methylumbelliferyl-phosphate. In an alternative probe system, the detection probes may be a fluorescent compound, such as fluorescein, phycobiliprotein, rhodamine and their derivatives and analogs. Other suitable detection probes may be described in U.S. Pat. No. 5,670,381 to Jou, et al. and U.S. Pat. No. 5,252,459 to Tarcha. et al., which are incorporated herein in their entirety by reference thereto for all purposes.

The detection probes may be used alone or in conjunction with particle, such as described above. For example, in one embodiment, latex particles are utilized that are labeled with a fluorescent or colored dye. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc.

In some instances, it is desired to modify the detection probes in some manner so that they are more readily able to bind to the analyte. In such instances, the detection probes may be modified with certain specific binding members that are adhered thereto to form conjugated probes. Specific binding members generally refer to a member of a specific binding pair, i.e., two different molecules where one of the molecules chemically and/or physically binds to the second molecule. For instance, immunoreactive specific binding members may include antigens, haptens, aptamers, antibodies (primary or secondary), and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin (or derivatives thereof), biotin and streptavidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte, i.e., an analyte-analog, may be used so long as it has at least one epitope in common with the analyte.

The specific binding members may generally be attached to the detection probes using any of a variety of well-known techniques. For instance, covalent attachment of the specific binding members to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the detection probe may contain a relatively high surface concentration of polar groups. In addition, although detection probes are often functionalized after synthesis, in certain cases, such as poly(thiophenol), the particles are capable of direct covalent linking with a protein without the need for further modification. For example, one embodiment of the present invention for covalently conjugating a particle-containing detection probe involves first activating carboxylic groups on the probe surface using carbodiimide. In the second step, the activated carboxylic acid groups are reacted with an amino group of an antibody to form an amide bond. The activation and/or antibody coupling may occur in a buffer, such as phosphate-buffered saline (PBS) (e.g., pH of 7.2) or 2-(N-morpholino) ethane sulfonic acid (MES) (e.g., pH of 5.3). The resulting detection probes may then be blocked with ethanolamine, for instance, to block any remaining activated sites. Overall, this process forms a conjugated detection probe, where the antibody is covalently attached to the probe. Besides covalent bonding, other attachment techniques, such as physical adsorption, may also be utilized in the present invention.

Referring again to FIG. 2, the porous membrane 123 defines a first detection 131 for detecting the presence of amines and a corresponding control zone 132, such as described above. In addition, the porous membrane 123 also defines a second detection zone 135 to detect the presence of an analyte within the test sample. The detection zone 135 may be positioned downstream or upstream from the detection zone 131.

The detection zone 135 may contain an immobilized capture reagent that is generally capable of forming a chemical or physical bond with detection probes or complexes thereof. In some embodiments, the capture reagent may be a biological capture reagent. Such biological capture reagents are well known in the art and may include, but are not limited to, antigens, haptens, protein A or G, neutravidin, avidin, streptavidin, captavidin, antibodies (e.g., polyclonal, monoclonal, etc.), and complexes thereof. The immobilized capture reagents serve as stationary binding sites for probe conjugate/analyte complexes. In some instances, the analytes, such as antibodies, antigens, etc., have two binding sites. Upon reaching the detection zone 135, one of these binding sites is occupied by the specific binding member of the complexed detection probes. However, the free binding site of the analyte may bind to the immobilized capture reagent. Upon being bound to the immobilized capture reagent, the complexed detection probes form a new ternary sandwich complex.

Similar to the detection zone 131, the detection zone 135 may also provide any number of distinct detection regions so that a user may better determine the concentration of a particular analyte within a test sample. Each region may contain the same capture reagents, or may contain different capture reagents for capturing multiple analytes. For example, the detection zone 135 may include two or more distinct detection regions (e.g., lines, dots, etc.). The detection regions may be disposed in the form of lines in a direction that is substantially perpendicular to the flow of the test sample through the assay device 120. Likewise, in some embodiments, the detection regions may be disposed in the form of lines in a direction that is substantially parallel to the flow of the test sample through the assay device.

Although the second detection zone 135 provides accurate results for detecting an analyte, it is sometimes difficult to determine the relative concentration of the analyte within the test sample under actual test conditions. Thus, the assay device 120 may also include a calibration zone 137. In this embodiment, the calibration zone 137 is formed on the porous membrane 123 and is positioned downstream from the second detection zone 135. Alternatively, however, the calibration zone 137 may also be positioned upstream from the detection zone 135. The calibration zone 137 may be provided with a capture reagent that is capable of binding to calibration probes or uncomplexed detection probes that pass through the length of the membrane 123. When utilized, the calibration probes may be formed from the same or different materials as the detection probes. Generally speaking, the calibration probes are selected in such a manner that they do not bind to the capture reagent at the detection zone 135.

The capture reagent of the calibration zone 137 may be the same or different than the capture reagent used in the detection zone 135. For example, in one embodiment, the capture reagent is a biological capture reagent. In addition, it may also be desired to utilize various non-biological materials for the capture reagent of the calibration zone 137. The polyelectrolytes may have a net positive or negative charge, as well as a net charge that is generally neutral. For instance, some suitable examples of polyelectrolytes having a net positive charge include, but are not limited to, polylysine (commercially available from Sigma-Aldrich Chemical Co., Inc. of St. Louis, Mo.), polyethyleneimine; epichlorohydrin-functionalized polyamines and/or polyamidoamines, such as poly(dimethylamine-co-epichlorohydrin); polydiallyldimethyl-ammonium chloride; cationic cellulose derivatives, such as cellulose copolymers or cellulose derivatives grafted with a quaternary ammonium water-soluble monomer; and so forth. In one particular embodiment, CelQuat® SC-230M or H-100 (available from National Starch & Chemical, Inc.), which are cellulosic derivatives containing a quaternary ammonium water-soluble monomer, may be utilized. Moreover, some suitable examples of polyelectrolytes having a net negative charge include, but are not limited to, polyacrylic acids, such as poly(ethylene-co-methacrylic acid, sodium salt), and so forth. It should also be understood that other polyelectrolytes may also be utilized, such as amphiphilic polyelectrolytes (i.e., having polar and non-polar portions). For instance, some examples of suitable amphiphilic polyelectrolytes include, but are not limited to, poly(styryl-B-N-methyl 2-vinyl pyridinum iodide) and poly(styryl-b-acrylic acid), both of which are available from Polymer Source, Inc. of Dorval, Canada. Further examples of internal calibration systems that utilize polyelectrolytes are described in more detail in U.S. Patent App. Publication No. 2003/0124739 to Song, et al., which is incorporated herein in it entirety by reference thereto for all purposes.

Regardless of the capture reagent utilized, the calibration zone 137 may be used to calibrate the signal intensity of the detection zone 135 under different assay conditions. For example, the detection and calibration signals may be plotted versus analyte concentration for a range of known analyte concentrations to generate a calibration curve. To determine the quantity of analyte in an unknown test sample, the signal ratio may then be converted to analyte concentration according to the calibration curve. It should be noted that any appropriate mathematical relationship may be plotted versus the analyte concentration to generate the calibration curve.

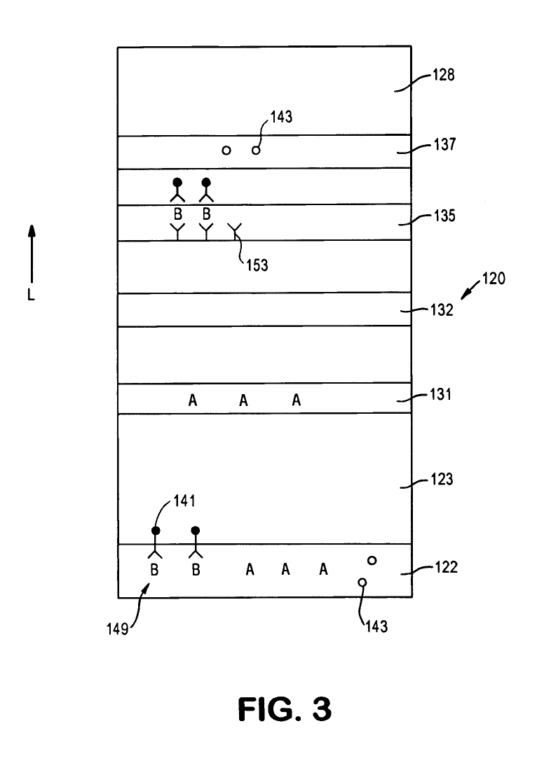

Referring to FIG. 3, one embodiment of a method for simultaneously detecting the presence of amines and an analyte within a test sample using the assay device 120 will now be described in more detail. Initially, a test sample containing amines “A” and an analyte “B” is applied to the sample pad (not shown) and travels in the direction “L” to the conjugate pad 122, where the analyte B mixes with detection probes 141 conjugated with an antibody and calibration probes 143 (may or may not be conjugated). The analyte B binds with the conjugated detection probes 141 to form analyte/conjugated probe complexes 149. These complexes 149 travel on to the second detection zone 135 and bind to an antibody 153. Finally, the calibration probes 143 travel through both the detection zone 135 to bind with a polyelectrolyte (not shown) at the calibration zone 137. Once captured, the intensity of the signal of the detection probes 141 may be determined (visually or with instrumentation) at the second detection zone 135. In addition, the intensity of the signal of the calibration probes 143 may also be measured at the calibration zone 137. The absolute amount of the analyte may be ascertained by comparing the signal intensity at the detection zone 131 with the signal intensity at the calibration zone 137.

Simultaneously, amines A from the test sample also react with the chemichromic dye (not shown) present at the first detection zone 131. After the reaction, the first detection zone 131 changes color. Thus, the color intensity of the dye at the first detection zone 131 may be determined (visually or with instrumentation). In addition, the color intensity of the dye may also be measured at the control zone 132, which is substantially constant relative to the color intensity of the reacted dye at the detection zone 131. The absolute amount of the amines A may then be ascertained by comparing the color intensity at the first detection zone 131 with the color intensity at the control zone 132.

Although various assay device configuration have been described herein, it should be understood that any known assay device may be utilized that is capable of incorporating a chemichromic dye in accordance with the present invention. For example, besides flow-through devices that utilize a porous membrane as a fluidic medium, such as described above, an assay device that utilizes one or more fluidic channels as a fluidic medium for the test sample may also be used in the present invention. Likewise, other detection techniques may be used for determining the presence of an analyte within the test sample. For example, electrochemical affinity assays may also be utilized, which detect an electrochemical reaction between an analyte (or complex thereof) and a capture ligand on an electrode strip. For example, various electrochemical assays are described in U.S. Pat. No. 5,508,171 to Walling, et al.; U.S. Pat. No. 5,534,132 to Vreeke, et al.; U.S. Pat. No. 6,241,863 to Monbouguette; U.S. Pat. No. 6,270,637 to Crismore, et al.; U.S. Pat. No. 6,281,006 to Heller, et al.; and U.S. Pat. No. 6,461,496 to Feldman, et al., which are incorporated herein in their entirety by reference thereto for all purposes.

In addition, it should be understood that both sandwich and competitive assay formats may be used to detect an analyte according to the present invention. Techniques and configurations of sandwich and competitive assay formats are well known to those skilled in the art. For example, sandwich formats assay formats typically involve mixing the test sample with antibodies to the analyte. These antibodies are mobile and linked to the label. This mixture is then contacted with a chromatographic medium containing a band or zone of immobilized antibodies to the analyte. The chromatographic medium is often in the form of a strip resembling a dipstick. When the complex of the analyte and the labeled antibody reaches the zone of the immobilized antibodies on the chromatographic medium, binding occurs and the bound labeled antibodies are localized at the zone. This indicates the presence of the analyte. This technique may be used to obtain quantitative or semi-quantitative results. Some examples of such sandwich-type assays are described by U.S. Pat. No. 4,168,146 to Grubb, et al. and U.S. Pat. No. 4,366,241 to Tom, et al., which are incorporated herein in their entirety by reference thereto for all purposes. In a competitive assay, the probe is generally a labeled analyte or analyte-analog that competes for binding of an antibody with any unlabeled analyte present in the sample. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler, et al., which are incorporated herein in their entirety by reference thereto for all purposes. Various other device configurations and/or assay formats are also described in U.S. Pat. No. 5,395,754 to Lambotte, et al.; U.S. Pat. No. 5,670,381 to Jou, et al.; and U.S. Pat. No. 6,194,220 to Malick, et al., which are incorporated herein in their entirety by reference thereto for all purposes.

The present invention provides a relatively simple, compact and cost-efficient device for accurately detecting amines and optionally other analytes within a test sample (e.g., vaginal fluid). The test result may be visible so that it is readily observed by the person performing the test in a prompt manner and under test conditions conducive to highly reliable and consistent test results. The device may then be discarded as a unit when the test is concluded.

The present invention may be better understood with reference to the following examples.

### EXAMPLE 1

The ability of alpha-naphtholbenzein to indicate the presence of an amine was demonstrated. 50-microliter solutions were provided that contained varying concentrations of putrescine (Sigma-Aldrich Chemical Company of Milwaukee, Wis., USA, 99% pure) in acetonitrile, i.e., 0.0, 0.15, 0.30, 0.60, 1.25, 2.50, 5.00, and 10.00 milligrams of putrescine per milliliter (which corresponds to 0; 37.5; 75; 150; 300; 600; 1,200; and 2,400 ppm, respectively). These solutions were placed in a microtiter plate well and mixed with 150 microliters of a solution containing alpha-naphtholbenzein (Sigma-Aldrich Chemical Company) in acetonitrile. Three (3) alpha-naphtholbenzein concentrations were tested, i.e., 0.01, 0.05, and 0.10 milligrams per milliliter. Upon mixing, the wells were then incubated at room temperature for less than 1 minute. Absorbance readings were then measured at a wavelength of 650 nanometers using a microplate reader from Dynex Technologies of Chantilly, Virginia (Model # MRX). The results are shown in FIG. 4. As indicated, the dye readily detected the presence of putrescine. Further, the level of detection sensitivity was readily controlled by varying the dye concentration.

### EXAMPLE 2

The ability of alpha-naphtholbenzein to indicate the presence of an amine was demonstrated. 50-microliter solutions were provided that contained varying concentrations of cadaverine (Sigma-Aldrich Chemical Company of Milwaukee, Wis., USA, 95% pure) in acetonitrile, i.e., 0.0, 0.15, 0.30, 0.60, 1.25, 2.50, 5.00, and 10.00 milligrams of cadaverine per milliliter (which corresponds to 0; 37.5; 75; 150; 300; 600; 1,200; and 2,400 ppm, respectively). These solutions were placed in a microtiter plate well and mixed with 150 microliters of a solution containing alpha-naphtholbenzein (Sigma-Aldrich Chemical Company) in acetonitrile. Three (3) alpha-naphtholbenzein concentrations were tested, i.e., 0.01, 0.05, and 0.10 milligrams per milliliter. Upon mixing, the wells were then incubated at room temperature for less than 1 minute. Absorbance readings were then measured at a wavelength of 650 nanometers using a microplate reader from Dynex Technologies of Chantilly, Virginia (Model # MRX). The results are shown in FIG. 5. As indicated, the dye readily detected the presence of cadaverine. Further, the level of detection sensitivity was readily controlled by varying the dye concentration.

### EXAMPLE 3

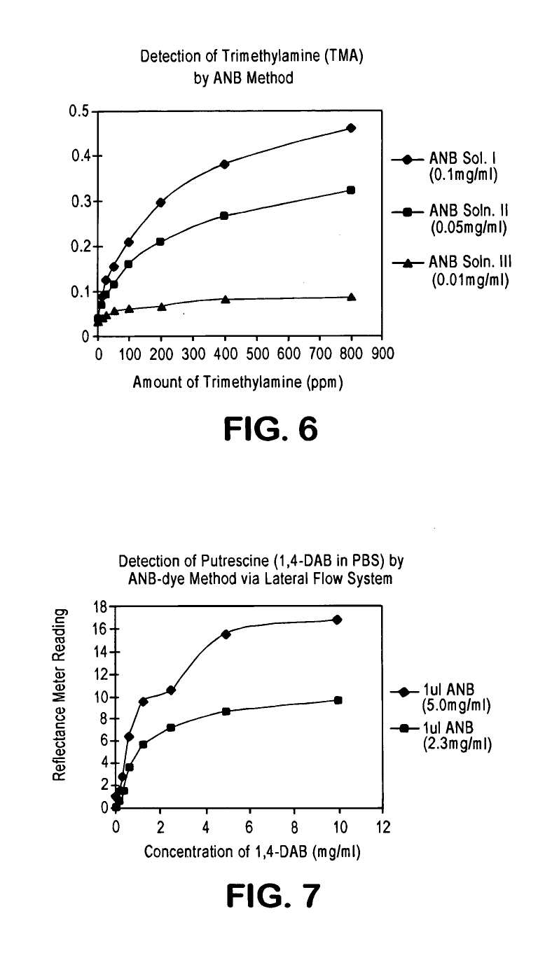

The ability of alpha-naphtholbenzein to indicate the presence of an amine was demonstrated. 50-microliter solutions were provided that contained varying concentrations of trimethylamine (Sigma-Aldrich Chemical Company of Milwaukee, Wis., USA, 40 wt. %) in water, i.e., 0.00, 0.25, 0.50, 1.00, 2.00, 4.00, 8,00, and 16.00 milligrams of trimethylamine per milliliter (which corresponds to 0; 12.5; 25; 50; 100; 200; 400; and 800 ppm, respectively). These solutions were placed in a microtiter plate well and mixed with 150 microliters of a solution containing alpha-naphtholbenzein (Sigma-Aldrich Chemical Company) in acetonitrile. Three (3) alpha-naphtholbenzein concentrations were tested, i.e., 0.01, 0.05, and 0.10 milligrams per milliliter. Upon mixing, the wells were then incubated at room temperature for less than 1 minute. Absorbance readings were then measured at a wavelength of 650 nanometers using a microplate reader from Dynex Technologies of Chantilly, Virginia (Model # MRX). The results are shown in FIG. 6. As indicated, the dye readily detected the presence of trimethylamine. Further, the level of detection sensitivity was readily controlled by varying the dye concentration.

### EXAMPLE 4

The ability to form a lateral flow assay device with multiple detection zones was demonstrated. Three sets of assay device samples were prepared (Samples 1-3). Each assay device sample was formed from a nitrocellulose porous membrane (HF 12002 from Millipore, Inc.) having a length of approximately 30 centimeters laminated onto a corresponding supporting card. Chemichromic detection zones were formed on each of the samples using two different stock solutions of alpha-naphtholbenzein (Sigma-Aldrich Chemical Company), each of which had a volume of 6200 microliters and contained a methanol/water solvent (4/6 ratio). The first stock solution contained alpha-naphtholbenzein in a concentration of 5.0 milligrams per milliliter, while the second stock solution contained alpha-naphtholbenzein in a concentration of 2.3 milligrams per milliliter. One (1) microliter of these stock solutions was then stripped onto the samples to form the chemichromic detection zones. In addition, monoclonal antibody for C-reactive protein (CRP Mab2) (A#5804, available from BiosPacific, Inc., concentration of 1 milligram per milliliter) was immobilized downstream from the chemichromic detection zone on the porous membrane to form the other detection zone. The samples were then dried for 1 hour at a temperature of 37° C.

After forming the assay devices, Sample 1 was then applied with 40 microliters of a solution containing 2.5 milligrams of putrescine, 0.5 milligrams of C-reactive protein (CRP), and 10 microliters of gold particles conjugated with C-reactive protein (Mab1) (A#5811, available from BiosPacific, Inc.) in a PBS buffer. Sample 2 was applied with 40 microliters of a solution containing 2.5 milligrams of putrescine and 0.5 milligrams of CRP in a PBS buffer. Finally, Sample 3 was applied with 40 microliters of a solution containing 0.5 milligrams of CRP in a PBS buffer. The assay devices were visually observed for the detection signal intensity. For Sample 3, the chemichromic dye remained an orange color and the CRP-detection zone exhibited no color. For Sample 2, the chemichromic dye changed from an orange color to a gray color, but the CRP-detection zone still exhibited no color. Finally, for Sample 1, the chemichromic dye changed from an orange color to a gray color and the CRP-detection zone exhibited a red color.

### EXAMPLE 5

The ability to detect an amine using a lateral flow assay device was demonstrated. Assay device samples were prepared as described above in Example 4. Thereafter, solutions were provided that contained varying concentrations of putrescine (Sigma-Aldrich Chemical Company of Milwaukee, Wis., USA, 99% pure) in acetonitrile, i.e., 0.0, 0.15, 0.30, 0.60, 1.25, 2.50, 5.00, and 10.00 milligrams of putrescine per milliliter. These solutions were applied to the samples, and reflectance readings were then measured for the samples as shown in FIG. 7. As indicated, the dye readily detected the presence of putrescine. Further, the level of detection sensitivity was readily controlled by varying the dye concentration.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. An assay device for detecting the presence or absence of amines within a test sample, said assay device comprising a fluidic medium that defines a detection zone, wherein a chemichromic dye is contained within said detection zone, said chemichromic dye being capable of undergoing a detectable color change upon reaction with one or more amines.

2. An assay device as defined in claim 1, wherein said chemichromic dye is an arylmethane.

3. An assay device as defined in claim 2, wherein said arylmethane is selected from the group consisting of diarylmethanes and triarylmethanes.

4. An assay device as defined in claim 2, wherein said chemichromic dye is a triarylmethane having the following general structure: 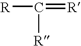
wherein R, R′, and R″ are independently selected from substituted and unsubstituted aryl groups.

5. An assay device as defined in claim 4, wherein said aryl groups are phenyl groups, naphthyl groups, or anthracenyl groups.

6. An assay device as defined in claim 5, wherein at least one of said aryl groups is amino-substituted, hydroxyl-substituted, carboxyl-substituted, sulfonic-substituted, alkyl-substituted, carbonyl-substituted, or combinations thereof.

7. An assay device as defined in claim 4, wherein said triarylmethane is pararosanilin, alpha-naphtholbenzein, naphthocrome green, or analogs thereof.

8. An assay device as defined in claim 3, wherein said chemichromic dye is a diarylmethane.

9. An assay device as defined in claim 8, wherein said diarylmethane is 4,4′-bis (dimethylamino) benzhydrol or analogs thereof.

10. An assay device as defined in claim 1, wherein said fluidic medium is a porous membrane.

11. An assay device as defined in claim 1, wherein said fluidic medium includes at least one flow channel.

12. An assay device as defined in claim 1, wherein said fluidic medium is in fluid communication with detection probes.

13. An assay device as defined in claim 12, wherein said detection probes are conjugated with a specific binding member for the analyte.

14. An assay device as defined in claim 13, wherein said fluidic medium defines a second detection zone within which is immobilized a capture reagent, said capture reagent being configured to bind to said detection probes or complexes thereof to generate a detection signal, wherein the amount of an analyte in the test sample is proportional to the intensity of said detection signal.

15. An assay device as defined in claim 1, wherein said fluidic medium further defines a control zone within which a chemichromic dye is contained, said control zone being located downstream from said detection zone.

16. An assay device for detecting the presence or absence of both amines and an analyte within a test sample, said assay device comprising a porous membrane that is in fluid communication with detection probes conjugated with a specific binding for the analyte, said porous membrane defining:
a first detection zone within which a triarylmethane dye is immobilized, said triarylmethane dye being capable of undergoing a detectable color change upon reaction with one or more amines; and
a second detection zone within which a capture reagent is immobilized, said capture reagent being configured to bind to said detection probes or complexes thereof to generate a detection signal, wherein the amount of an analyte in the test sample is proportional to the intensity of said detection signal.

17. An assay device as defined in claim 16, wherein said triarylmethane has the following general structure: 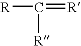
wherein R, R′, and R″ are independently selected from substituted and unsubstituted aryl groups.

18. An assay device as defined in claim 17, wherein said aryl groups are phenyl groups, naphthyl groups, or anthracenyl groups.

19. An assay device as defined in claim 18, wherein at least one of said aryl groups is amino-substituted, hydroxyl-substituted, carboxyl-substituted, alkyl-substituted, sulfonic-substituted, carbonyl-substituted, or combinations thereof.

20. An assay device as defined in claim 16, wherein said triarylmethane is pararosanilin, alpha-naphtholbenzein, naphthocrome green, or analogs thereof.

21. An assay device as defined in claim 16, wherein said porous membrane further defines a control zone within which a chemichromic dye is contained, said control zone being located downstream from said detection zone.

22. A method for detecting the presence or absence of amines within a test sample, said method comprising:
i) contacting an assay device with a test sample containing one or more amines, said assay device comprising a fluidic medium that defines a detection zone, wherein a chemichromic dye is contained within said detection zone that undergoes a color change upon reacting with said amines; and
ii) measuring the color intensity of said chemichromic dye at said detection zone after reacting with said amines, wherein said color intensity corresponds to a certain concentration of said amines within the test sample.

23. A method as defined in claim 22, further comprising comparing the measured color intensity with a color intensity of a chemichromic dye that is not reacted with amines.

24. A method as defined in claim 23, wherein said chemichromic dye that is not reacted with amines is contained within a control zone, said control zone being defined by said fluidic medium and being located downstream from said detection zone.

25. A method as defined in claim 22, wherein said chemichromic dye is an arylmethane.

26. A method as defined in claim 25, wherein said chemichromic dye is a triarylmethane having the following general structure: 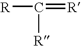
wherein R, R′, and R″ are independently selected from substituted and unsubstituted phenyl groups, naphthyl groups, and anthracenyl groups.

27. A method as defined in claim 26, wherein at least one of R, R′, or R″ is amino-substituted, hydroxyl-substituted, carboxyl-substituted, alkyl-substituted, carbonyl-substituted, sulfonic-substituted, or combinations thereof.

28. A method as defined in claim 26, wherein said triarylmethane is pararosanilin, alpha-naphtholbenzein, naphthocrome green, or analogs thereof.

29. A method as defined in claim 22, wherein said fluidic medium is a porous membrane.

30. A method as defined in claim 22, wherein said fluidic medium is in fluid communication with detection probes conjugated with a specific binding member for the analyte.

31. A method as defined in claim 22, wherein said fluidic medium defines a second detection zone within which a capture reagent is immobilized, said second detection zone being configured to generate a detection signal.

32. A method as defined in claim 31, further comprising measuring the intensity of said detection signal, wherein the amount of an analyte in the test sample is proportional to the intensity of said detection signal.

33. A method as defined in claim 32, wherein said fluidic medium defines a calibration zone that is configured to generate a calibration signal.

34. A method as defined in claim 33, further comprising calibrating the intensity of said detection signal with the intensity of said calibration signal.

35. A method as defined in claim 22, wherein the presence of said amines in the test sample reflects the presence of infection.

36. A method as defined in claim 35, wherein the test sample is obtained from vaginal fluid.

37. A method as defined in claim 35, wherein the test sample is obtained from a wound exudate.

38. A method as defined in claim 35, wherein the test sample is obtained from food.
